(19) United States
(12) Patent Application Publication
(10) Pub. No.: US 2010/0241225 A1
(43) Pub. Date: Sep. 23, 2010

(54) CURABLE RESIN COMPOSITION FOR INTRAOCULAR LENS, INTRACULAR LENS MATERIAL AND INTRACULAR LENS

(75) Inventor: SATORU YAMADA, SHIZUOKA (JP)

Correspondence Address: YOUNG & THOMPSON, 209 Madison Street, Suite 500, Alexandria, VA, 22314, US

(73) Assignee: FUJIFILM CORPORATION, TOKYO (JP)

(21) Appl. No.: 12/729,371

(22) Filed: Mar. 23, 2010

(30) Foreign Application Priority Data
Mar. 23, 2009 (JP) 2009-069933

(51) Int. Cl. A61F 2/16; C08F 28/06

(52) U.S. Cl. 623/6.38; 526/257; 526/256

(57) ABSTRACT

A curable resin composition for intraocular lens is provided, the curable resin composition including: (a) a first monomer selected from compounds represented by formula (I); (b) a polyfunctional second monomer; and (c) a radical polymerization initiator:

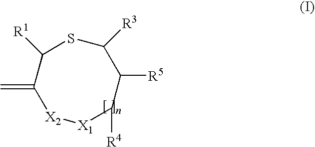

wherein R¹ represents a hydrogen atom or an alkyl group; X₁ represents an oxygen atom or a sulfur atom; when X₁ represents a sulfur atom, X₂ represents CR² where R² represents a hydrogen atom or an alkyl group; when X₁ represents an oxygen atom, X₂ represents C═O; R³ to R⁵ each represents a hydrogen atom, an alkyl group, an aryl group, a hetero ring group, an alkoxy group, an aryloxy group, an alkylthio group, an arylthio group, an alkoxycarbonyl group, an aryloxycarbonyl group, an acyloxy group, an arylcarbonyloxy group, an acylamino group, a sulfonylamino group, an amino group, an acyl group or a halogen atom; and n represents 0 or 1.

### BACKGROUND OF THE INVENTION

1. Field of the Invention

The present invention relates to a cyclic allylsulfide compound-containing curable resin composition used for intraocular lenses, and further to intraocular lens materials and intraocular lenses which are made from such a curable resin composition.

2. Description of the Related Art

In treatment for a cataract, it is prevailingly performed that an intraocular lens is inserted after removal of a crystalline lens having clouded over. In recent years, soft intraocular lenses allowing insertion through small incisions by use of soft materials as materials for making the optic parts of intraocular lenses have come to be widely used in eye surgical procedures.

These soft intraocular lenses are required to have excellent transparency and flexibility. The materials known as intraocular lens materials which excel in transparency and flexibility include the copolymer of a mixture containing a (meth)acrylate monomer and a cross-linkable monomer (see JP-A-4-292609), the copolymer made by subjecting a monomer mixture containing 2-hydroxy-3-phenoxypropylacrylate as a monomeric ingredient and a cross-linking agent to copolymerization (see JP-A-8-173522), and so on.

All the intraocular lenses formed from the materials disclosed in those documents have comparatively excellent transparency and flexibility. However, such intraocular lenses have a problem of causing the phenomenon referred to as “glistening”. The term “glistening” means a phenomenon that the transparency of an intraocular lens is seriously lowered or lost by water drops accumulated inside the material of the optic part after the intraocular lens is implanted in a patient's eye.

As materials capable of solving this glistening problem, the copolymer using as main constituent monomers an arylacrylic hydrophobic monomer and a hydrophilic monomer (see JP-A-2001-316426), the polymer made by polymerizing a hydrophilic monomer, such as a hydroxyl group-containing alkyl(meth)acrylate, a (meth)acrylamide monomer or an N-vinyl lactam (see JP-A-11-56998), and so on are disclosed. In addition, the copolymer of a monomer mixture, which contains a (meth)acrylate monomer of specific structure, and a cross-linkable monomer (see JP-A-2006-249381) is disclosed as a material which can reduce glistening and has a high refractive index and excellent flexibility.

As another intraocular lens material, the copolymer made from a monomer mixture containing a monofunctional (meth)acrylate monomer, a bifunctional (meth)acrylate monomer and an aromatic functional (meth)acrylate monomer (see JP-T-2008-543432, the term “JP-T” as used herein means a published Japanese translation of a PCT patent application) is disclosed. By using the material disclosed in JP-T-2008-543432, intraocular lenses high in strength and refractive index and excellent in flexibility can be obtained.

As mentioned above, compounds having general-purpose polymerizable groups such as an acryloyl group and a methacryloyl group have so far been used for intraocular lenses. However, these materials bring about a great curing shrinkage in volume, so there is a problem in controlling their shapes. In addition, when the polymers disclosed in JP-A-2001-316426, JP-A-11-56998, JP-A-2006-249381 and JP-T-2008-543432 are used in intraocular lenses, they have problems in terms of lens's appearance and the like, because the intraocular lenses made from them become cloudy through the immersion in water, and from a glistening viewpoint. So, it is desired to introduce improvements to those polymers.

Meanwhile, as monomers which can contribute to a small curing shrinkage in volume, the cyclic allylsulfide monomers are disclosed (see Macromolecules, 1994, 27, 7935, Macromolecules, 1996, 29, 6983, Macromolecules, 2000, 33, 6722, J. Polym. Sci.: Part A Polym. Chem., 2001, 39, 202, Japanese Patent No. 3299542 and Japanese Patent No. 4153031). In these documents, though there are descriptions on the adhesive use, dental use and lens use possibility of those monomers, any description about the effect that those monomers can be used for intraocular lenses is not present at all.

### SUMMARY OF THE INVENTION

Although various arts have been developed as mention above, it is desired to pursue further development of intraocular lenses which have high refractive indexes, excellent flexibility and transparency, and cause no change in their appearances even under the water, and are prevented from causing glistening, and undergo slight curing shrinkages in their volumes.

Objects of the invention is therefore to provide a material for intraocular lenses, which can ensure a high refractive index, excellent flexibility, excellent transparency, causes no change in appearance even under the water, and can prevent occurrence of glistening, and to provide intraocular lenses using such a material.

As a result of our intensive studies for attaining the above objects, the following aspects have been found. Specifically, it has been found that, in polymerization of methacrylate or acrylate monomers, which are radical polymerizable monomers for general purpose use, their substituents are introduced onto every two carbon atoms as side chains of the polymers produced because their polymerization is an addition reaction to vinyl groups; as a result, the polymers have rises in Tg and flexibility of lenses made from the polymers, or the ability of lenses to be bent, is impaired.

In contrast to those monomers, the use of cyclic allylsulfide monomers represented by the formula (I) as illustrated hereinafter leads to introduction of one substituent per 8-atom link including carbon and sulfur atoms in the case of polymerization using e.g. an 8-membered cyclic allylsulfide monomer; as a result, Tg rises can be reduced and flexibility can be ensured. On these points, our attention has been focused. In addition, the carbon-sulfur-carbon bond is long in bonding length and small in bonding angle, so the cyclic allylsulfide monomers can form polymers which are flexible in themselves. Moreover, the cyclic allylsulfide monomers have high refractive indexes in comparison with acrylate polymers currently in use, because they have sulfur atoms in their main chains.

And it has been found that intraocular lens materials having excellent flexibility and high refractive indexes and ensuring suitable use as soft intraocular lenses can be provided by using cyclic allylsulfide monomers represented by the formula (I) as (a) a first monomer in combination with (b) a polyfunctional second monomer and (c) a radical polymerization initiator. Add to this, it also has been found that formation of intraocular lenses by use of these intraocular lens materials allows prevention of the glistening occurring in the intraocular lenses after they are implanted in patients' eyes, though the action mechanism of such prevention remains uncertain. Moreover, it has been found that curable resin compositions prepared from the foregoing combinations for use in intraocular lenses undergo small curing shrinkage in volume and the products formed by curing them can be reduced in deformation; as a result, they can ensure very high dimensional stability and high-level shape control. Thus, the invention has come to be achieved.

More specifically, the problems of the invention can be solved by the following aspects.

(1) A curable resin composition for intraocular lens, including:

(a) at least one first monomer selected from compounds represented by following formula (I);

(b) a polyfunctional second monomer; and

(c) a radical polymerization initiator:

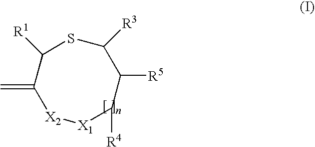

wherein R¹ represents a hydrogen atom or an alkyl group;

X₁ represents an oxygen atom or a sulfur atom;

when X₁ represents a sulfur atom, X₂ represents CR² where R² represents a hydrogen atom or an alkyl group;

when X₁ represents an oxygen atom, X₂ represents C═O;

R³ to R⁵ each independently represents a hydrogen atom, an alkyl group, an aryl group, a hetero ring group, an alkoxy group, an aryloxy group, an alkylthio group, an arylthio group, an alkoxycarbonyl group, an aryloxycarbonyl group, an acyloxy group, an arylcarbonyloxy group, an acylamino group, a sulfonylamino group, an amino group, an acyl group or a halogen atom; and

n represents 0 or 1.

(2) The curable resin composition for intraocular lens as described in (1),

wherein, in the formula (I),

X₁ represents a sulfur atom;

X₂ represents CR²; and

n represents 1.

(3) The curable resin composition for intraocular lens as described in (1) or (2),

wherein the polyfunctional second monomer (b) is at least one selected from the group consisting of compounds represented by following formula (AI), polyfunctional methacrylate monomers and polyfunctional acrylate monomers:

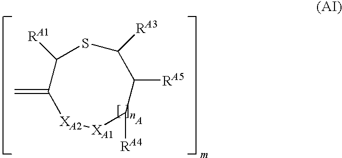

wherein R^{A1} represents a hydrogen atom or an alkyl group;

X_{A1} represents an oxygen atom or a sulfur atom;

when X_{A1} represents a sulfur atom, X_{A2} represents CR^{A2} where R^{A2} represents a hydrogen atom or an alkyl group;

when X_{A1} represents an oxygen atom, X_{A2} represents C═O;

R^{A3} to R^{A5} each independently represents a hydrogen atom, an alkyl group, an aryl group, a hetero ring group, an alkoxy group, an aryloxy group, an alkylthio group, an arylthio group, an alkoxycarbonyl group, an aryloxycarbonyl group, an acyloxy group, an arylcarbonyloxy group, an acylamino group, a sulfonylamino group, an amino group, an acyl group or a halogen atom;

n_{A} represents 0 or 1;

m represents 2 to 6; and

the formula (AI) represents a dimer to hexamer which bonds via any of R^{A1} to R^{A5}.

(4) The curable resin composition for intraocular lens as described in any one of (1) to (3), which contains the polyfunctional second monomer (b) in an amount of from 0.05 to 40 mass % with respect to a total amount of monomers contained in the curable resin composition.

(5) The curable resin composition for intraocular lens as described in any one of (1) to (4), further including:

a monomer having ultraviolet absorbing power in an amount of from 0.05 to 8 mass % with respect to a total amount of monomers contained in the curable resin composition.

(6) The curable resin composition for intraocular lens as described in any one of (1) to (5), further including:

a monomer having yellow coloring power in an amount of 0.0001 to 0.5 mass % with respect to a total amount of monomers contained in the curable resin composition.

(7) An intraocular lens material obtained by polymerization reaction of the curable resin composition for intraocular lens as described in any one of (1) to (6).

(8) An intraocular lens, including:

an optic part; and

a supporting part that fixes the optic part to an appropriate place in the eye,

wherein the optic part is formed from the intraocular lens material as described in (7).

### BRIEF DESCRIPTION OF THE DRAWINGS

The features of the invention will appear more fully upon consideration of the exemplary embodiments of the inventions, which are schematically set forth in the drawings, in which:

FIG. 1 is a schematic diagram which illustrates one embodiment of intraocular lens relating to the invention; and

FIG. 2 is a schematic diagram which illustrates another embodiment of intraocular lens relating to the invention.

### DETAILED DESCRIPTION OF THE INVENTION

### Curable Resin Composition for Intraocular Lens

The present curable resin composition used for intraocular lenses contains (a) at least one first monomer selected from compounds represented by the following formula (I); (b) a polyfunctional second monomer; and (c) a radical polymerization initiator.

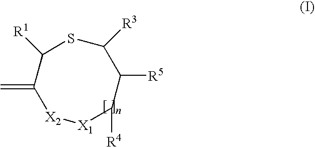

In the formula (I), R¹ represents a hydrogen atom or an alkyl group; X₁ represents an oxygen atom or a sulfur atom; when X₁ represents a sulfur atom, X₂ represents CR² where R² represents a hydrogen atom or an alkyl group; when X₁ represents an oxygen atom, X₂ represents C═O; R³ to R⁵ each independently represents a hydrogen atom, an alkyl group, an aryl group, a hetero ring group, an alkoxy group, an aryloxy group, an alkylthio group, an arylthio group, an alkoxycarbonyl group, an aryloxycarbonyl group, an acyloxy group, an arylcarbonyloxy group, an acylamino group, a sulfonylamino group, an amino group, an acyl group or a halogen atom; and n represents 0 or 1.

The term “a curable resin composition” as used in the invention refers to a composition which can produce polymerization reaction of polymerizable ingredients contained therein by irradiation with light or radiation, application of heat, use of a radical polymerization initiator, or so on.

### (a) Compound Represented by Formula (I):

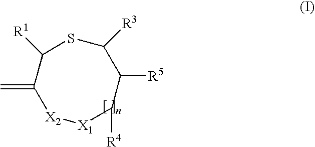

In the formula (I), R¹ represents a hydrogen atom or an alkyl group; X₁ represents an oxygen atom or a sulfur atom; when X₁ represents a sulfur atom, X₂ represents CR² where R² represents a hydrogen atom or an alkyl group; when X₁ represents an oxygen atom, X₂ represents C═O; R³ to R⁵ each independently represents a hydrogen atom, an alkyl group, an aryl group, a hetero ring group, an alkoxy group, an aryloxy group, an alkylthio group, an arylthio group, an alkoxycarbonyl group, an aryloxycarbonyl group, an acyloxy group, an arylcarbonyloxy group, an acylamino group, a sulfonylamino group, an amino group, an acyl group or a halogen atom; and n represents 0 or 1.

The compounds represented by the formula (I) in the invention are monofunctional cyclic allylsulfide monomers. The term “monofunctional cyclic allylsulfide monomers” as used herein refers to the cyclic allylsulfide monomers which each have one structure corresponding to the formula (I) (or the formula (II) or (III) illustrated hereinafter) in one molecule thereof.

Hereafter, the compounds represented by the formula (I) are referred to as monofunctional cyclic allylsulfide monomers or monofunctional cyclic allylsulfide compounds. The monofunctional cyclic allylsulfide monomers represented by the formula (I) can function as polymerizable ingredients. More specifically, these monomers initiate radical polymerization directly or with the aid of the action of a radical polymerization initiator by at least one of heating and irradiating with light and, by undergoing ring-opening polymerization, they are converted into polymers having double bonds. The polymerization can be expressed in the following reaction scheme:

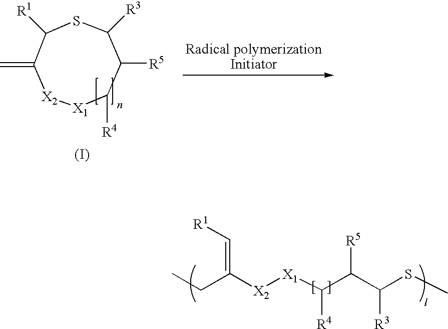

In the above reaction scheme, R¹ represents a hydrogen atom or an alkyl group; X₁ represents an oxygen atom or a sulfur atom; when X₁ represents a sulfur atom, X₂ represents CR² where R² represents a hydrogen atom or an alkyl group; when X₁ represents an oxygen atom, X₂ represents C═O; R³ to R⁵ each independently represents a hydrogen atom, an alkyl group, an aryl group, a hetero ring group, an alkoxy group, an aryloxy group, an alkylthio group, an arylthio group, an alkoxycarbonyl group, an aryloxycarbonyl group, an acyloxy group, an arylcarbonyloxy group, an acylamino group, a sulfonylamino group, an amino group, an acyl group or a halogen atom; and n represents 0 or 1. 1 represents the number of repeating units.

Polymerization of a methacrylate monomer or an acrylate monomer, which has so far been used as a general-purpose radical polymerizable monomer, is inhibited to some extent by dissolved oxygen. In contrast to these monomers, the monofunctional cyclic allylsulfide monomers represented by the formula (I) have a feature that their polymerization does not undergo the oxygen inhibition as seen in thiol-ene reaction, because their growth radicals are sulfur radicals and they have hydrogen atoms on the α-positions of their respective sulfur atoms (on the positions of carbon atoms adjacent to their respective sulfur atoms). Therefore, the polymerization reaction in the case of using the cyclic allylsulfide monomers is more likely to proceed even in the presence of oxygen or dissolved oxygen and more resistant to oxygen inhibition than in the case of using general-purpose monomers; as a result, curing can be achieved by use of a reduced amount of radical polymerization initiator, speeding-up of resin curing at the time of lens making becomes possible, and the productivity can be increased.

In addition, because polymerization of a methacrylate or arylate monomer, which is a general-purpose radical polymerizable monomer, is the reaction taking place through addition to the vinyl group, the substituent group as the side chain is introduced into the resultant polymer every two carbon atoms. This causes a rise in Tg and impairs flexibility of being able to be bent. As to the monofunctional cyclic allylsulfide monomers represented by the formula (I), on the other hand, one substituent group is introduced every one linkage group having, say, eight atoms including carbon, sulfur and other atoms in the case of an eight-membered cyclic allylsulfide monomer. As a result, the rise in Tg can be reduced and the flexibility can be assured. Moreover, the carbon-to-sulfur-to-carbon bond has a long bond length and a small bonding angle, so a polymer which in itself has flexibility can be formed. Consequently, the intraocular lens materials formed from the curable resin compositions according to the invention have excellent flexibility and can be suitably used for soft intraocular lenses allowing insertion into the eye through a small incision.

In addition, the intraocular lens materials formed from the curable resin composition for intraocular lenses according to the invention have higher refractive indexes than those formed from (meth)acrylate polymers currently in use, because they contain sulfur atoms in their respective main chains. Therefore, reductions in thickness and weight can be planned for the lenses to be formed.

Further, the intraocular lens materials are high in transparency. And what's more, glistening occurring after implantation of an intraocular lens in the eye can be reduced when the lens is formed with such a polymeric material, though the action mechanism thereof is uncertain.

Furthermore, since such a polymeric material has a small curing shrinkage in volume and can reduce deformation of the cured material it forms, it is very high in dimensional stability and allows sophisticated shape control. When intraocular lenses are those of diffraction type in particular, fine structures are required to be formed therein, so the present intraocular lens materials are used to advantage.

In the formula (I), R¹ represents a hydrogen atom or an alkyl group. The alkyl group represented by R¹ may be linear in form or may have a branch, and it may be an unsubstituted one or may have a substituent. The number of carbon atoms in the alkyl group is preferably from 1 to 20, far preferably from 1 to 10, particularly preferably from 1 to 3. Additionally, the term “number of carbon atoms” as used in the invention with regard to a certain group having a substituent means the number of carbon atoms in the substituent-free moiety of the group.

Examples of the alkyl group represented by R¹ to R⁵ include a methyl group, an ethyl group, a normal propyl group, an isopropyl group, a normal butyl group, an isobutyl group, a tertiary butyl group, a pentyl group, a cyclopentyl group, a hexyl group, a cyclohexyl group, a heptyl group, an octyl group, a tertiary octyl group, a 2-ethylhexyl group, a decyl group, a dodecyl group, an octadecyl group, a 2,3-dibromopropyl group, an adamantyl group, a benzyl group and a 4-bromobenzyl group. These groups each may further have a substituent.

The aryl group represented by each of R³, R⁴ and R⁵ in the formula (I) may be unsubstituted one, or may have a substituent. The number of carbon atoms contained in such an aryl group is preferably from 6 to 30, particularly preferably from 6 to 20. Examples of such an aryl group include a phenyl group, a naphthyl group and an anthranyl group. These groups may further have substituents.

The heterocyclic group represented by each of R³, R⁴ and R⁵ in the formula (I) may be unsubstituted one, or may have a substituent. Such a heterocyclic group is preferably a heterocyclic group having 4 to 14 carbon atoms, far preferably a heterocyclic group having 4 to 10 carbon atoms, particularly preferably a heterocyclic group having 5 carbon atoms. Examples of the heterocyclic group represented by each of R³, R⁴ and R⁵ include groups derived from a pyridine ring, a piperazine ring, a thiophene ring, a pyrrole ring, an imidazole ring, an oxazole ring and a thiazole ring, respectively. These groups may further have substituents. Of those heterocyclic rings, a pyridine ring in particular is preferred over the others.

In the formula (I), the alkoxy group represented by each of R³, R⁴ and R⁵ may be linear in form or may have a branch, and it may be unsubstituted one or may have a substituent. The number of carbon atoms in such an alkoxy group is preferably from 1 to 30, far preferably from 1 to 20. Examples of such an alkoxy group include a methoxy group, an ethoxy group, a normal propyloxy group, an isopropyloxy group, a normal butyloxy group, an isobutyloxy group, a tertiary butyloxy group, a pentyloxy group, a cyclopentyloxy group, a hexyloxy group, a cyclohexyloxy group, a heptyloxy group, an octyloxy group, a tertiary octyloxy group, a 2-ethylhexyloxy group, a decyloxy group, a dodecyloxy group, an octadecyloxy group, a 2,3-dibromopropyloxy group, an adamantyloxy group, a benzyloxy group and a 4-bromobenzyloxy group.

In the formula (I), the aryloxy group represented by each of R³, R⁴ and R⁵ may be unsubstituted one or may have a substituent. The number of carbon atoms in such an aryloxy group is preferably from 6 to 30, particularly preferably from 6 to 20. Examples of such an aryloxy group include a phenyloxy group, a naphthyloxy group and an anthranyloxy group.

In the formula (I), the alkylthio group represented by each of R³, R⁴ and R⁵ may be linear in form or may have a branch, and it may be unsubstituted one or may have a substituent. The number of carbon atoms in such an alkylthio group is preferably from 1 to 30, far preferably from 1 to 20. Examples of such an alkylthio group include a methylthio group, an ethylthio group, a normal propylthio group, an isopropylthio group, a normal butylthio group, an isobutylthio group, a tertiary butylthio group, a pentylthio group, a cyclopentylthio group, a hexylthio group, a cyclohexylthio group, a heptylthio group, an octylthio group, a tertiary octylthio group, a 2-ethylhexylthio group, a decylthio group, a dodecylthio group, an octadecylthio group, a 2,3-dibromopropylthio group, an adamantylthio group, a benzylthio group and a 4-bromobenzylthio group.

In the formula (I), the arylthio group represented by each of R³, R⁴ and R⁵ may be unsubstituted one or may have a substituent. The number of carbon atoms in such an arylthio group is preferably from 6 to 30, particularly preferably from 6 to 20. Examples of such an arylthio group include a phenylthio group, a naphthylthio group and an anthranylthio group.

In the formula (I), the alkoxycarbonyl group represented by each of R³, R⁴ and R⁵ may be unsubstituted one or may have a substituent. The number of carbon atoms in such an alkoxycarbonyl group is preferably from 2 to 30, far preferably from 2 to 20. Examples of such an alkoxycarbonyl group include a methyloxycarbonyl group, an ethyloxycarbonyl group, a normal propyloxycarbonyl group, an isopropyloxycarbonyl group, a normal butyloxycarbonyl group, an isobutyloxycarbonyl group, a tertiary butyloxycarbonyl group, a pentyloxycarbonyl group, a cyclopentyloxycarbonyl group, a hexyloxycarbonyl group, a cyclohexyloxycarbonyl group, a heptyloxycarbonyl group, an octyloxycarbonyl group, a tertiary octyloxycarbonyl group, a 2-ethylhexyloxycarbonyl group, a decyloxycarbonyl group and a dodecyloxycarbonyl group.

In the formula (I), the aryloxycarbonyl group represented by each of R³, R⁴ and R⁵ may be unsubstituted one or may have a substituent. The number of carbon atoms in such an aryloxycarbonyl group is preferably from 7 to 30, particularly preferably from 7 to 20. Examples of such an aryloxycarbonyl group include a phenyloxycarbonyl group, a naphthyloxycarbonyl group and an anthranyloxycarbonyl group.

In the formula (I), the acyloxy group represented by each of R³, R⁴ and R⁵ may be unsubstituted one or may have a substituent. The number of carbon atoms in such an acyloxy group is preferably from 2 to 30, particularly preferably from 2 to 20. Examples of such an acyloxy group include a methylcarbonyloxy group, an ethylcarbonyloxy group, a normal propylcarbonyloxy group, an isopropylcarbonyloxy group, a normal butylcarbonyloxy group, an isobutylcarbonyloxy group, a tertiary butylcarbonyloxy group, a pentylcarbonyloxy group, a cyclopentylcarbonyloxy group, a hexylcarbonyloxy group, a cyclohexylcarbonyloxy group, a heptylcarbonyloxy group, an octylcarbonyloxy group, a tertiary octylcarbonyloxy group, a 2-ethylhexylcarbonyloxy group, a decylcarbonyloxy group, a dodecylcarbonyloxy group and a benzoyloxy group.

In the formula (I), the arylcarbonyloxy group represented by each of R³, R⁴ and R⁵ may be unsubstituted one or may have a substituent. The number of carbon atoms in such an arylcarbonyloxy group is preferably from 7 to 30, particularly preferably from 7 to 20. Examples of such an arylcarbonyloxy group include a phenylcarbonyloxy group, a naphthylcarbonyloxy group and an anthranylcarbonyloxy group.

In the formula (I), the acylamino group represented by each of R³, R⁴ and R⁵ may be unsubstituted one or may have a substituent. The number of carbon atoms in such an acylamino group is preferably from 2 to 30, far preferably from 2 to 20. Examples of such an acylamino group include a methylcarbonylamino group, an ethylcarbonylamino group and a phenylcarbonylamino group.

In the formula (I), the sulfonylamino group represented by each of R³, R⁴ and R⁵ may be unsubstituted one or may have a substituent. The number of carbon atoms in such a sulfonylamino group is preferably from 1 to 30, far preferably from 1 to 20. Examples of such a sulfonylamino group include a methylsulfonylamino group, an ethylsulfonylamino group and a phenylsulfonylamino group.

In the formula (I), the amino group represented by each of R³, R⁴ and R⁵, though may be either monosubstituted or disubstituted one, is preferably a disubstituted amino group. These groups may further have substituents or needn't. The number of carbon atoms in such an amino group is preferably from 1 to 30, far preferably from 1 to 20. Examples of such an amino group include a dimethylamino group and a diphenylamino group.

In the formula (I), the acyl group represented by each of R³, R⁴ and R⁵ may be unsubstituted one or may have a substituent. The number of carbon atoms in such an acyl group is preferably from 2 to 30, far preferably from 2 to 20. Examples of such an acyl group include an acetyl group and a benzoyl group.

Examples of the halogen atom represented by each of R³, R⁴ and R⁵ in the formula (I) include a chloro group, a bromo group and an iodo group. Of these radicals, a bromo radical is preferred over the others.

Examples of a substituent by which each of the groups represented by R¹, R², R³, R⁴ and R⁵ in the formula (I) can further be substituted include a halogen atom, an alkyl group, an alkenyl group, an alkoxy group, an aryloxy group, an alkylthio group, an alkoxycarbonyl group, an aryloxycarbonyl group, an amino group, an acyl group, an alkylaminocarbonyl group, an arylaminocarbonyl group, a sulfonamido group, a cyano group, a carboxyl group, a hydroxyl group and a sulfonic acid group. Of these substituents, a halogen atom, an alkoxy group and an alkylthio group in particular are preferred over the others.

In the formula (I), n represents an integer of 0 or 1. n is preferably 1.

In the formula (I), R¹ represents a hydrogen atom or an alkyl group. It is preferable that R¹ represents a hydrogen atom.

In the formula (I), when X₁ represents a sulfur atom, X₂ represents CR² where R² represents a hydrogen atom or an alkyl group. When X₁ represents a sulfur atom, formula (I) is represented by the following formula (II). Further, in the formula (I), when X₁ represents an oxygen atom, X₂ represents C═O. When X₁ represents an oxygen atom, formula (I) is represented by the following formula (III).

The formulae (II) and (III) are illustrated below.

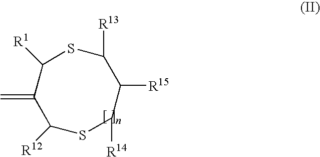

R¹ in the formula (II) has the same meaning as R¹ in the formula (I). R¹² represents a hydrogen atom or an alkyl group, each of R¹³, R¹⁴ and R¹⁵ has the same meaning as R³, R⁴ and R⁵ in the formula (I), and n represents 0 or 1.

In the formula (II), n represents an integer of 0 or 1. n is preferably 1.

R¹ and R¹² are independent of each other, and each is preferably a hydrogen atom or a methyl group, far preferably a hydrogen atom.

Preferred aspects of the compound represented by the formula (II) are e.g. as follows. Each of R¹ and R¹² independently represents a hydrogen atom or a methyl group, preferably a hydrogen atom. And each of R¹³, R¹⁴ and R¹⁵ independently represents a hydrogen atom, an alkyl group, an aryl group, an alkoxy group, an aryloxy group, an alkoxycarbonyl group, an aryloxycarbonyl group, an acyloxy group, an arylcarbonyloxy group, an acylamino group, an amino group or an acyl group.

R¹³ is preferably a hydrogen atom or an alkyl group, far preferably a hydrogen atom.

When two or more R¹⁴s are present, they are independent of one another, and each is preferably a hydrogen atom, an alkyl group, an alkoxy group, an acyloxy group or an arylcarbonyloxy group, far preferably a hydrogen atom or a methyl group, further preferably a hydrogen atom.

R¹⁵ is preferably a hydrogen atom, an alkyl group, an aryl group or an acyloxy group, far preferably an alkyl group or an acyloxy group, further preferably an acyloxy group.

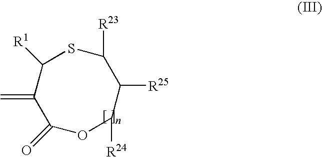

In the formula (III), R¹ has the same meaning as R¹ in the formula (I). Each of R²³, R²⁴ and R²⁵ has the same meaning as R³, R⁴ and R⁵ in the formula (I), and n represents 0 or 1.

In the formula (III), n represents an integer of 0 or 1. n is preferably 0.

R²³ is preferably a hydrogen atom or an alkyl group, far preferably a methyl group.

When two or more R²⁴s are present, they are independent of one another, and each is preferably a hydrogen atom, an alkyl group, an alkoxy group or an acyloxy group, far preferably a hydrogen atom or a methyl group, further preferably a hydrogen atom.

R²⁵ is preferably a hydrogen atom, an alkyl group, an aryl group, an acyloxy group or an arylcarbonyloxy group, far preferably a hydrogen atom, an alkyl group, an acyloxy group or an arylcarbonyloxy group, further preferably a hydrogen atom.

Examples of monofunctional cyclic allylsulfide compounds represented by the formula (I), and the formulae (II) and (III), are illustrated below. However, the invention should not be construed as being limited to these examples.

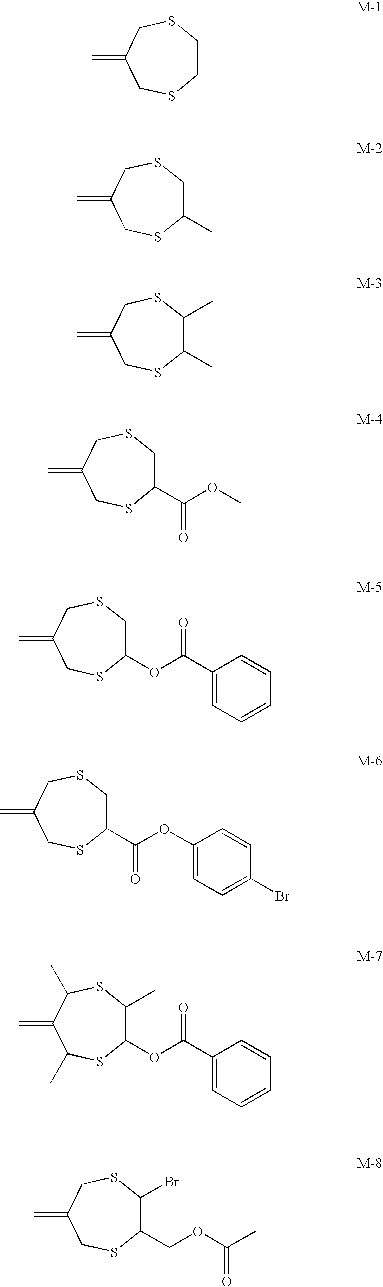

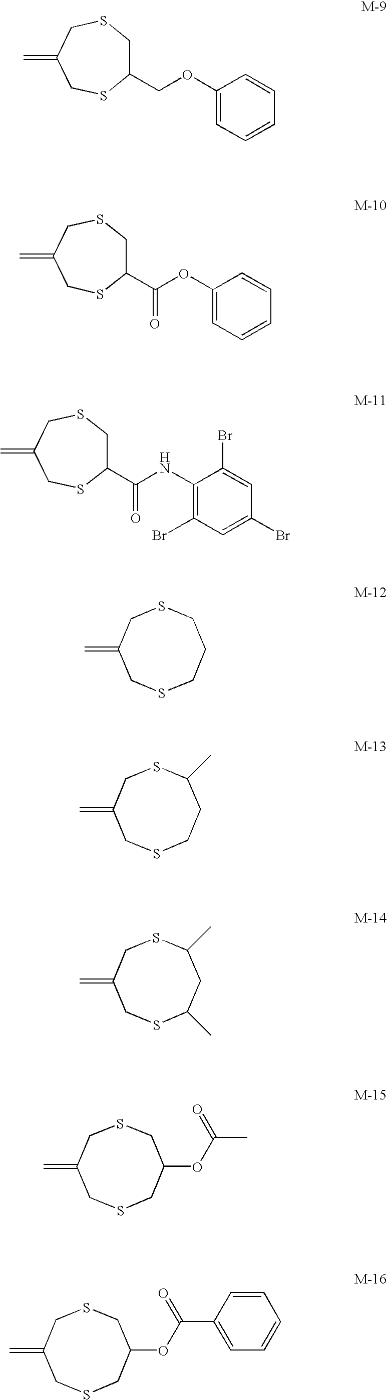

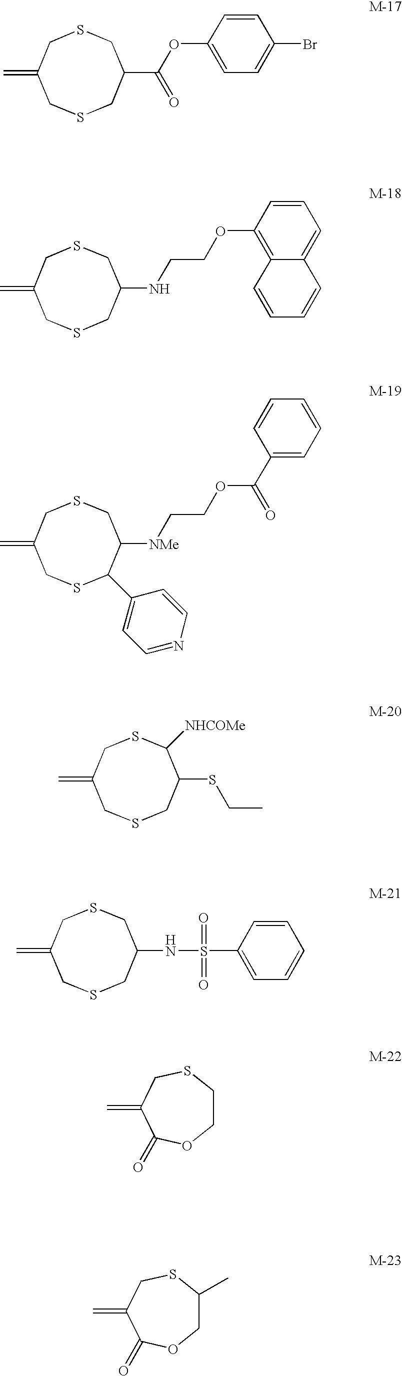

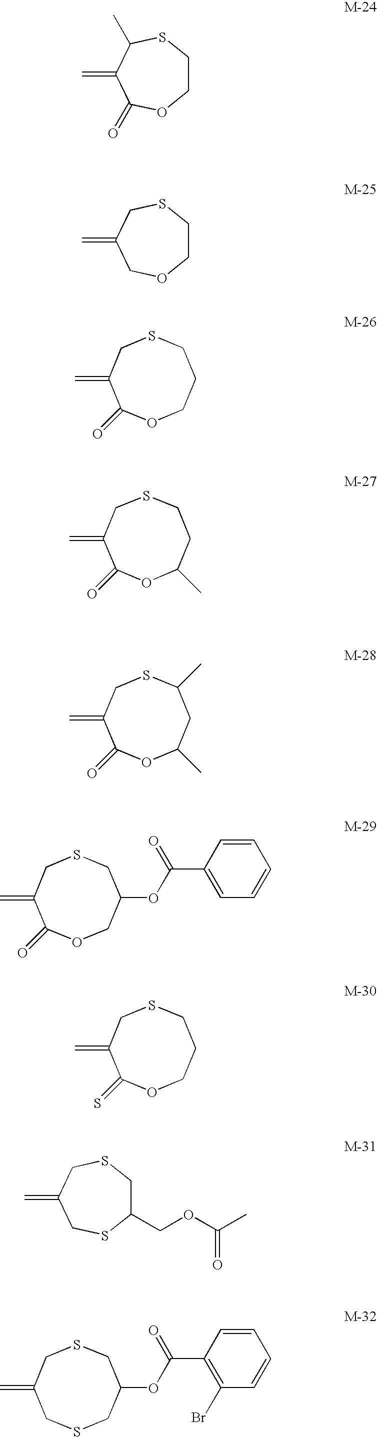

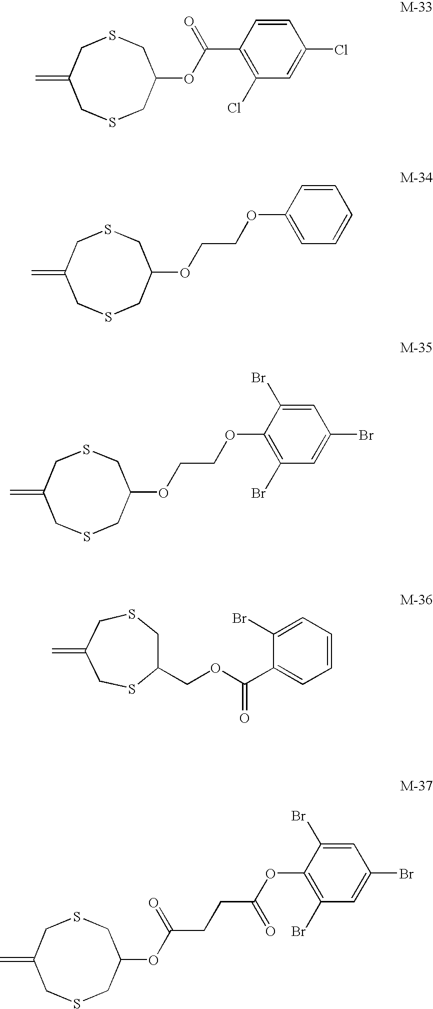

Synthesis methods of the compounds represented by the formulae (I) to (III) are described in detail e.g. in Macromolecules, 1994, 27, 7935; Macromolecules, 1996, 29, 6983; Macromolecules, 2000, 33, 6722; J. Polym. Sci.: Part A Polym. Chem., 2001, 39, 202; and so on.

The cyclic allylsulfide compounds may be used alone, or they can be used as combinations of two or more thereof.

The content of monofunctional cyclic allulsulfide monomers as the first monomer (a) has no particular limits, but it is preferably from 10 to 99 mass %, far preferably from 40 to 99 mass %, further preferably from 80 to 98 mass %, with respect to the total amount of the composition.

### (b) Polyfunctional Second Monomer:

The polyfunctional second monomer (b) contained in the curable composition of the present invention is illustrated below. As an example of the polyfunctional second monomer (b), compounds represented by the following formula (AI) or polyfunctional monomers other than the compounds represented by the formula (AI) (hereafter, the polyfunctional monomers other than the compounds represented by the formula (AI) are sometimes referred to as “other polyfunctional monomer”) can be given.

The compounds represented by the formula (AI) of the present invention are polyfunctional cyclic allylsulfide monomers. Hereafter, the compounds represented by the formula (AI) are referred to as polyfunctional cyclic allylsulfide monomers or polyfunctional cyclic allylsulfide compounds. Herein, the term polyfunctional cyclic allylsulfide monomer refers to the cyclic allylsulfide monomer which has two or more structures corresponding to the formula (I) (or the formula (II), or the formula (III)) per molecule thereof. The polyfunctional cyclic allylsulfide monomers represented by the formula (AI) can function as polymerizable ingredients like the monofunctional cyclic allylsulfide monomers represented by the formula (I).

The compounds represented by the formula (AI):

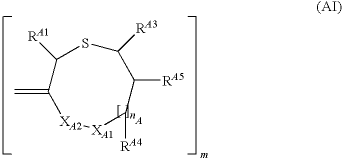

In the formula (AI), R^{A1} represents a hydrogen atom or an alkyl group; X_{A1} represents an oxygen atom or a sulfur atom; when X_{A1} represents a sulfur atom, X_{A2} represents CR^{A2} where R^{A2} represents a hydrogen atom or an alkyl group; when X_{A1} represents an oxygen atom, X_{A2} represents C═O; R^{A3} to R^{A5} each independently represents a hydrogen atom, an alkyl group, an aryl group, a hetero ring group, an alkoxy group, an aryloxy group, an alkylthio group, an arylthio group, an alkoxycarbonyl group, an aryloxycarbonyl group, an acyloxy group, an arylcarbonyloxy group, an acylamino group, a sulfonylamino group, an amino group, an acyl group or a halogen atom; n_{A} represents 0 or 1; and m represents 2 to 6. The formula (AI) represents a dimer to hexamer by bonding via any of R^{A1} to R^{A5}.

In the formula (AI), each of R^{A1} to R^{A5}, X_{A1}, X_{A2} and n_{A} has the same meaning as R¹ to R⁵, X₁, X₂ and n in the formula (I).

The formula (AI) forms a dimer to hexamer by bonding via any of R^{A1} to R^{A5}. It is preferable that the formula (AI) forms a dimer to hexamer by bonding via R^{A5}, more preferably forms a dimer to tetramer by bonding via R^{A5}.

It is preferable that the formula (AI) bonds via an arylcarbonyloxy group or an alkylcarbonyloxy group as R^{A1} and R^{A3} to R^{A5}, more preferably bonds via an arylcarbonyloxy group.

More specifically, single bond and the following linking groups (a) to (n) are given as R^{A1} and R^{A3} to R^{A5}.

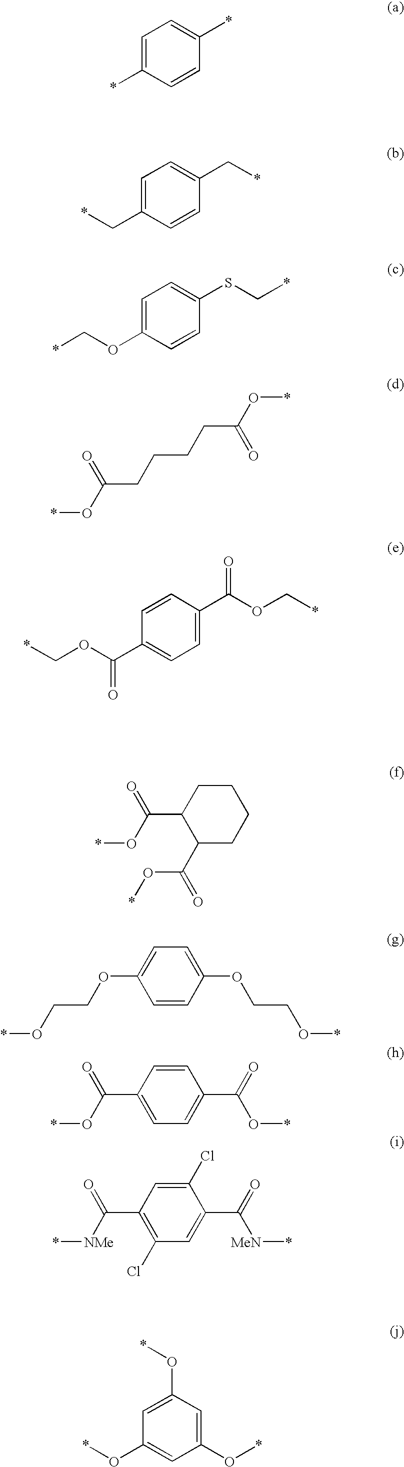

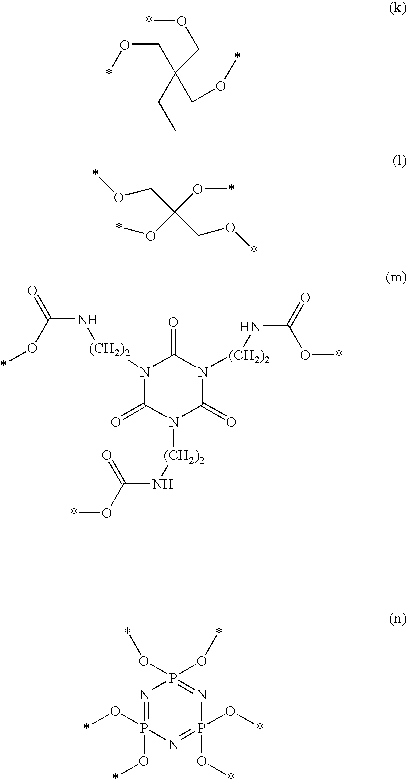

In the linking groups (a) to (n) shown above, * represents a linking site. Of these linking groups, (d), (e), (g) or (h) is preferable and (h) is more preferable, because there is an advantage that the shorter linking distance enables higher strength.

In the present invention, the combined use of the first monomer (a) and a compound represented by the formula (AI) as the second monomer (b) is a preferred form.

In the case of using a monofunctional cyclic allylsulfide monomer in combination with a polyfunctional cyclic allylsulfide monomer, the polyfunctional cyclic allylsulfide monomer functions as a cross-linking monomer. So, when wrought into lenses, the copolymer of those monomers can contribute to prevention of plastic deformation of lenses and further improvement in mechanical strength of lenses.

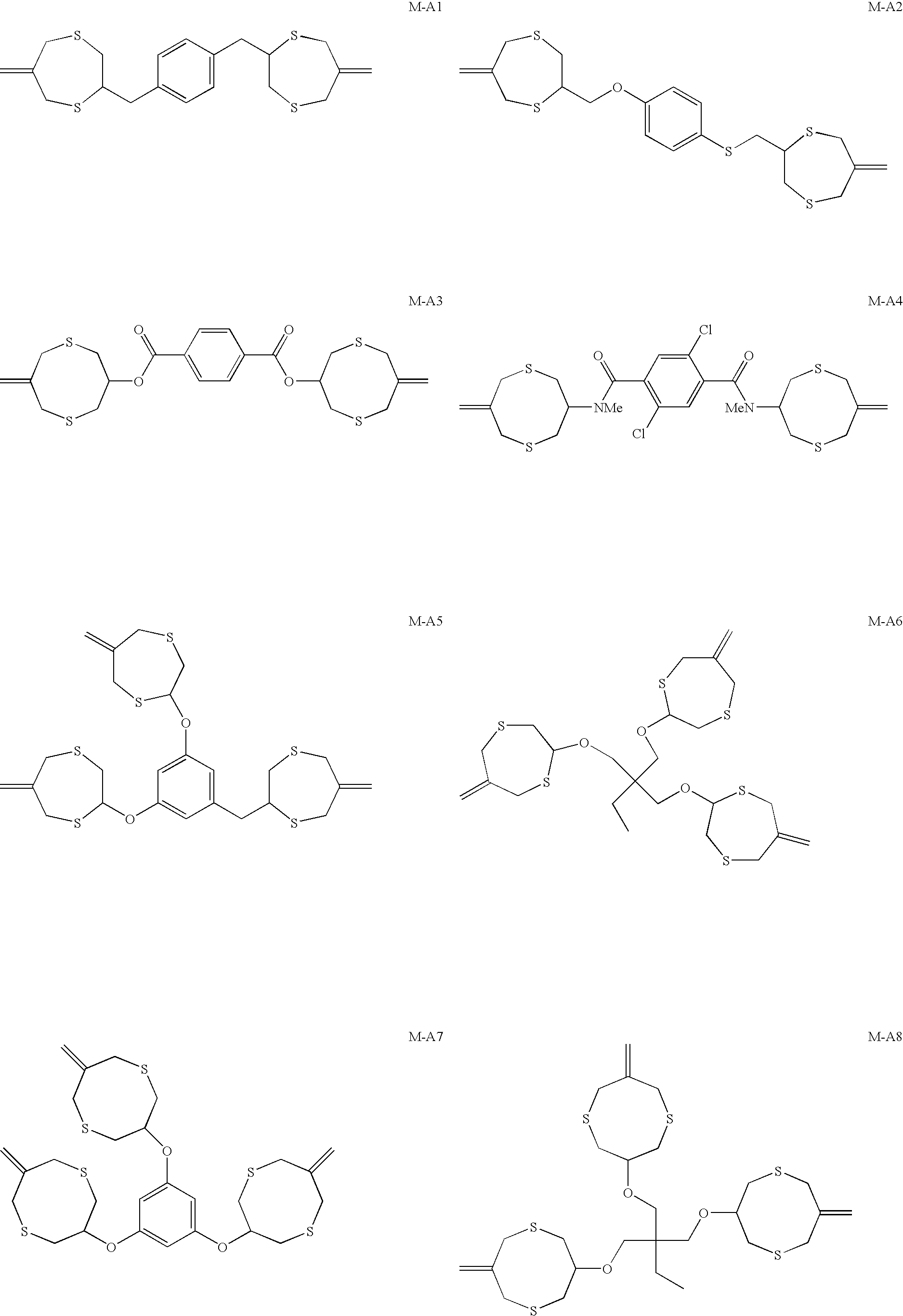

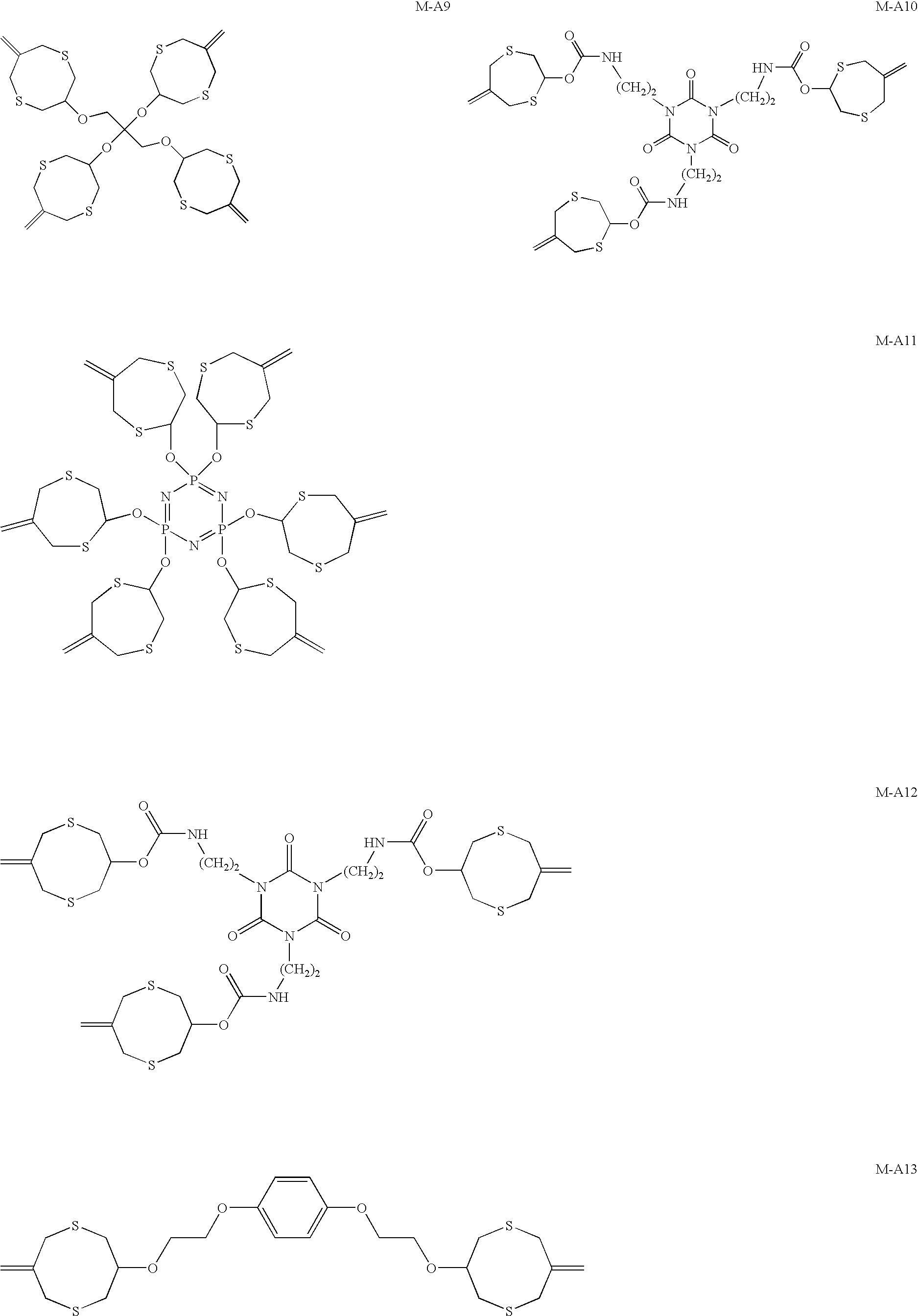

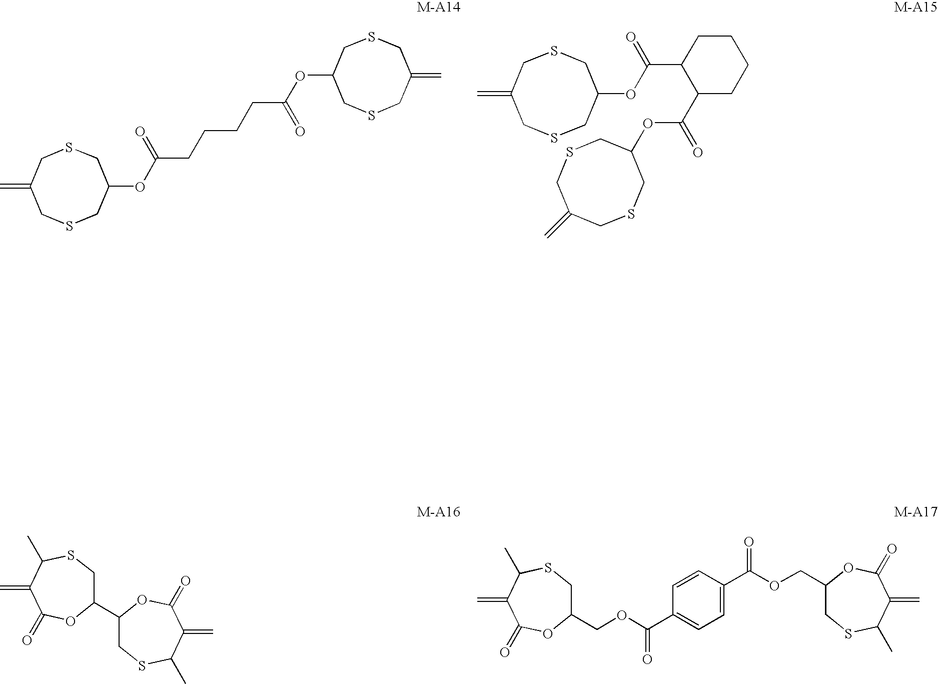

As other polyfunctional monomers, methacrylate monomers and acrylate monomers are suitable. And these methacrylate monomers and acrylate monomers are recognized as being copolymerizable with cyclic allylsulfide monomers. The polyfunctional second monomer (b) may be used alone, or they can be used as combinations of two or more thereof. By containing the first monomer (a) and the polyfunctional second monomer (b), the curable resin composition according to the invention can have an improved cross-linking density and can produce a copolymer resistant to destruction, so it can form intraocular lenses which are free of changes in appearances even under the water and protected against the glistening.

Polyfunctional (meth)acrylate monomers in particular are used suitably as other polyfunctional monomers of the polyfunctional second monomers. The term polyfunctional (meth)acrylate monomer refers to a (meth)acrylate monomer containing two or more acryloyl or methacryloyl groups in one molecule thereof. Examples of the polyfunctional (meth)acrylate monomer include 1,9-nonanediol di(meth)acrylate, ethylene glycol di(meth)acrylate, 2-hydroxy-1-acryloyloxy-3-methacryloxypropane, diethylene glycol di(meth)acrylate, triethylene glycol di(meth)acrylate, tetraethylene glycol di(meth)acrylate, 1,4-butanediol di(meth)acrylate and 1,6-hexanediol di(meth)acrylate.

Of these monomers, ethylene glycol di(meth)acrylate, triethylene glycol di(meth)acrylate, tetraethylene glycol di(meth)acrylate and 1,4-butanediol di(meth)acrylate are preferred over the others, and ethylene glycol di(meth)acrylate, triethylene glycol di(meth)acrylate and 1,4-butanediol di(meth)acrylate are far preferred.

As preferable combinations of a first monomer (a) and a polyfunctional second monomer (b), the following (1) to (3) are given:

(1) A monofunctional monomer represented by the formula (I) and a polyfunctional monomer other than the compounds represented by the formula (AI) (other polyfunctional monomer);

(2) A monofunctional monomer represented by the formula (I), a polyfunctional monomer represented by the formula (AI), and an other polyfunctional monomer; and

(3) A monofunctional monomer represented by the formula (I) and a polyfunctional monomer represented by the formula (AI).

Containing the foregoing (1) to (3), the present curable resin composition for intraocular lens can form intraocular lenses which have excellent flexibility and high refractive indexes, undergo no change in appearances even under the water and are prevented from developing the glistening.

The content of the polyfunctional second monomer (b) is preferably from 0.05 to 40 mass %, far preferably from 0.1 to 30 mass %, further preferably from 2 to 8 mass %, with respect to the total amount of the monomers contained in the curable resin composition of the present invention. When a polyfunctional monomer represented by the formula (AI) and an other polyfunctional monomer are used in combination as the polyfunctional second monomer (b), the polyfunctional monomer represented by the formula (AI) is preferably contained in an amount of from 50 to 90 mass %, far preferably from 60 to 85 mass %, further preferably from 65 to 80 mass %, with respect to the amount of the other polyfunctional monomer. By adjusting the content to the above range, the cured material of the composition can have compatibility between suitable mechanical strength and flexibility.

Moreover, it is preferable that the curable resin compositions according to the invention contain monomers having ultraviolet absorbing power.

The content of monomers having ultraviolet absorbing power is preferably from 0.05 to 8 mass %, far preferably from 3 to 6 mass %, with respect to the total amount of monomers in each composition. By adjusting the content of such monomers to the above range, adequate effect on UV protection can be achieved.

As the monomers having ultraviolet absorbing power, though any monomers can be used as long as they have ultraviolet absorbing power and can react with cyclic allylsulfide monomers, 2-(2′-hydroxy-3′-tetrabutyl-5′-methylophenyl)-5-(2′-methacryloxymethyl)benzotriazole in particular is suitable.

And it is also preferable that the curable resin compositions according to the invention contain monomers having yellow coloring power.

The content of monomers having yellow coloring power is preferably from 0.0001 to 0.5 mass %, far preferably from 0.0001 to 0.2 mass %, with respect to the total amount of monomers in each composition. Adjusting the content of such monomers to the range specified above allows patients having undergone surgery to be prevented from overly sensing blueness.

As the monomers having yellow coloring power, any monomers can be used as long as they have yellow coloring power and can react with cyclic allylsulfide monomers. As an example of such monomers, (4-(5-hydroxy-3-methyl-1-phenyl-4-pyrazolylmethylene)-3-methacrylamino-1-phenyl-2-pyrazoline-5-one) can be given.

### (c) Radical Polymerization Initiator

The present curable resin compositions contain radical polymerization initiators.

As to the radical polymerization initiators, there is no particular restriction, and any compounds can be used as long as they produce radicals when light, radiation or heat is applied thereto. The radical polymerization initiators for use in the present curable resin compositions can be chosen from publicly known ones as appropriate according to the natures of polymerizable compounds used in combination therewith and various characteristics of intended intraocular lenses.

Examples of such a radical polymerization initiator include azo-type initiators, such as 2,2′-azobisisobutyronitrile (AIBN), 2,2-azobis(4-methoxy-2,4-dimethylvaleronitrile), 2,2-azobis(2,4-dimethylvaleronitrile) (V-65), 2,2-azobis(2-methylpropionitrile), 2,2-azobis(2-methylbutyronitrile) and dimethyl 2,2′-Azobis(2-methylpropionate) (V-601), and organic peroxides, such as bis(4-t-butylcyclohexyl)peroxydicarbonate, benzoyl peroxide, 1,1,3,3-tetramethylbutyl hydroperoxide, t-hexyl hydroperoxide, t-butyl hydroperoxide and 3,5,5-trimethylhexanol peroxide. Of these compounds, AIBN, V-65 and V-601 are preferred over the others.

And examples of a photopolymerization initiator include methylorthobenzoyl benzoate, 1-hydroxy-cyclohexyl phenyl ketone, 2-hydroxy-2-methoxy-1-phenylpropane-1-one, 1-[4-(2-hydroxyethoxy)-phenyl]-2-hydroxy-2-methyl-1-propane-1-one and bis(2,4,6-trimethylbenzoyl)phenylphosphine oxide.

Polymerization initiators can be used alone or mixtures of two or more thereof. The polymerization initiator usage is preferably of the order of 0.1 to 5 mass % with respect to the total amount of monomers used.

### Intraocular Lens Material

Intraocular lens materials relating to the invention can be made by subjecting the present curable resin compositions for intraocular lens to polymerization reaction.

More specifically, monomer solutions containing the cyclic allylsulfide monomers and other polymerizable ingredients are each prepared, and then made to undergo polymerization reaction by being heated or irradiated with light to produce intraocular lens materials.

It is preferred that the monomer solutions prepared be made homogeneous by thorough stirring. The stirring can be carried out under conditions in usual methods.

In the case of adopting a thermal polymerization method, the reaction temperature can be chosen from a range of 40° C. to 120° C., preferably 80° C. to 100° C. The reaction time is preferably from 1 hour to 80 hours, far preferably from 2 hours to 48 hours. In the case of adopting a photopolymerization method, the reaction time is preferably from 1 minute to 3 hours, far preferably from 2 minutes to 2 hours.

The conclusion of polymerization reaction can be ascertained by subjecting the reaction product obtained to an extractive operation, such as supercritical extraction or solvent extraction, using a good solvent like acetone, methyl ethyl ketone or so on, and measuring the amount of unreacted monomer extraction by means of a gas chromatograph-mass spectrometer or the like.

When each of the present intraocular lens materials is a copolymer of two or more varieties of cyclic allysulfide monomers or a copolymer of cyclic allylsulfide monomer(s) and other monomer(s), the copolymer may be either a block copolymer or a random copolymer. In ordinary cases, the copolymer is a random copolymer.

It is appropriate that the present intraocular lens materials have properties which soft intraocular lenses as their best use should have. For instance, their appearance is preferably colorless and transparent. And their refractive indexes are preferably in a range of 1.50 to 1.65, far preferably in a range of 1.51 to 1.65.

### Intraocular Lens

Embodiments of the present intraocular lens are illustrated below with reference to drawings.

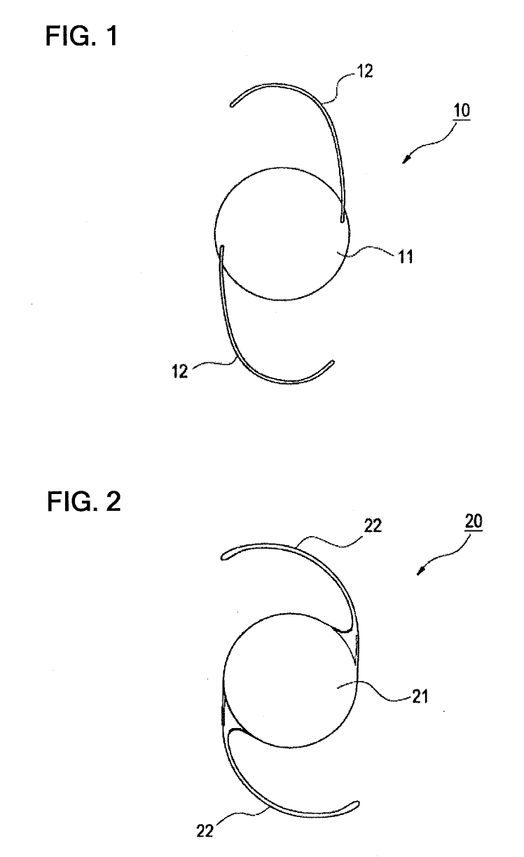

FIG. 1 is an oblique view of the intraocular lens concerning an embodiment of the invention. As shown in FIG. 1, the intraocular lens 10 concerning an embodiment of the invention has an optic part 11 of a specified refractory power and supporting parts 12 that fixes the optic part 11 to its proper place in the eye.

The optic part 11 is formed of the intraocular lens material made by polymerization reaction of the curable resin composition containing (a) at least one first monomer selected from compounds represented by the formula (I) mentioned above, (b) a polyfunctional second monomer and (c) a radical polymerization initiator.

The material for forming the supporting parts 12 has no particular restriction, and examples thereof include polypropylene, PMMA (polymethyl methacrylate) and polyimide. Alternatively, the supporting parts 12 can also be formed with an intraocular lens material which is the same as or different from the material forming the optic part 11.

The optic part 11 (as shown in FIG. 1) is not particularly restricted as to its making method, and it can be made in the usual way. In a preferred aspect, the making of the optic part is carried out by the method referred to as mold polymerization, wherein the polymerization reaction is performed in the interior of a mold for intraocular lenses. More specifically, a monomer solution as a raw material is charged into a mold having the shape corresponding to a shape of the optic part 11, and then pressurized as appropriate. Thus, both polymerization and molding are performed in one and the same mold, and thereby the optic part 11 can be formed.

In another making method, it is possible to form the optic part 11 having the desired shape by carrying out the polymerization reaction in the interior of an appropriate cast or vessel, thereby making a polymerized material shaped like a rod, block, plate or so on, and subjecting the polymerized material to cutting-out and abrading operations on a lathe and further to a polishing operation.

To the surface of the optic part 11, surface treatment, such as plasma treatment using argon, oxygen or nitrogen gas, may be given.

Intraocular lenses relating to the invention can be made by attaching separately-made supporting parts 12 to the optic part 11 made in the manner as mentioned above.

Although the optic part 11 and supporting parts 12 of the intraocular lens 10 are, as shown in FIG. 1, separate members in the embodiment illustrated above as an example, the present intraocular lenses are not limited to such a configuration, but the present intraocular lenses may have the configuration shown in FIG. 2, wherein the optic part 21 and the supporting parts 22 are formed in one piece. Additionally, the intraocular lens 20 having such a configuration can be made using a mold capable of integrally molding an optic part 21 and supporting parts 22.

The materials, forms, configurations, numbers, locations and other factors of various members exemplified in embodiments of the invention can be arbitrarily chosen and have no particular restrictions so long as they can ensure achievement of the invention.

### EXAMPLES

The invention will now be illustrated in more detail by reference to the following examples and comparative examples. Additionally, the invention should not be construed as being limited to the following examples in any way.

### Synthesis Examples of Compounds Represented by Formula (I)

Exemplified Compounds M-15, M-16 and M-24 were synthesized under the following reaction schemes (where R was a methyl group in the syntheses of M-15, a phenyl group in the synthesis of M-16, and a hydrogen atom in the syntheses of M-24) according to the method described in J. Polym. Sci.: Part A Polym. Chem., 2001, 39, 202. These exemplified compounds can be synthesized in similar manners by the changing of starting materials. And cyclic allylsulfide compounds having various substituents can be synthesized by the changing of substituents in starting materials.

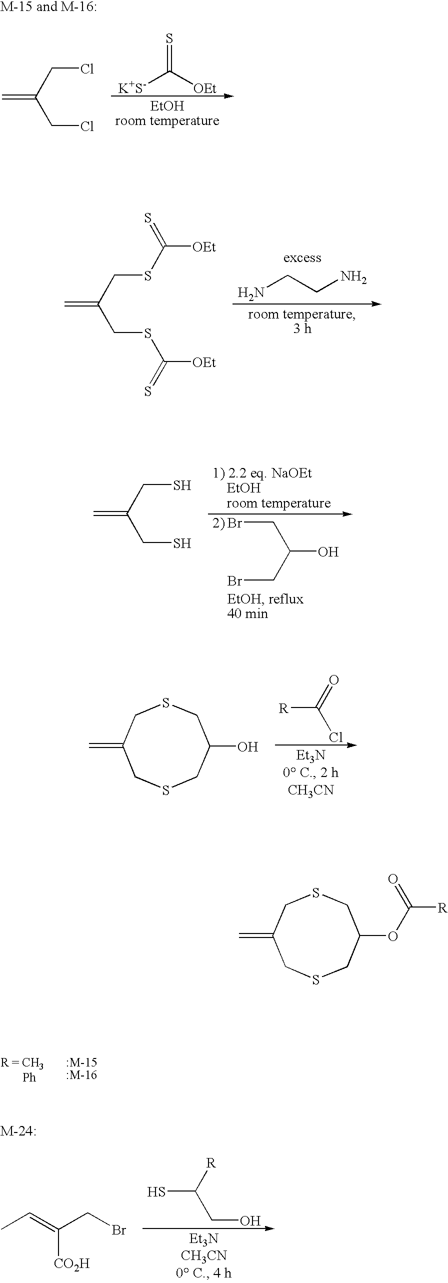

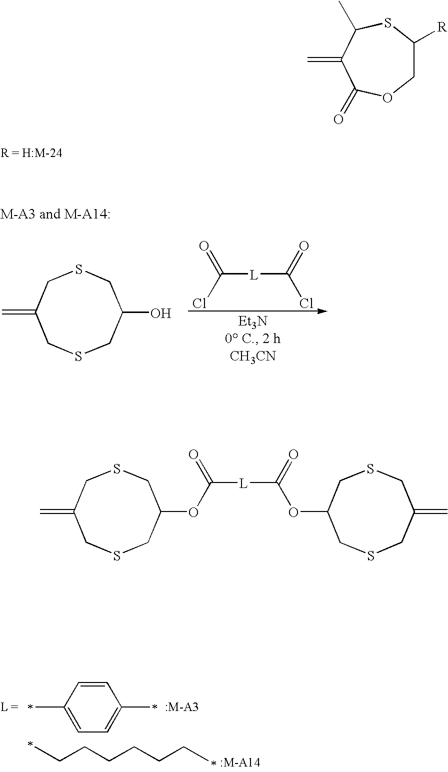

Physical data obtained are described below.

M-15: ¹H NMR (300 MHz, CDCl₃) δ2.00 (s, 3H), 3.05 (m, 4H), 3.21 (s, 4H), 4.95 (m, 1H), 5.20 (s, 2H)

M-16: ¹H NMR (300 MHz, CDCl₃) δ3.16 (d, 2H), 3.18 (d, 2H), 3.23 (s, 4H), 5.20 (s, 2H), 5.20 (m, 1H), 7.30 (m, 3H), 8.0 (m, 2H)

M-24: ¹H NMR (300 MHz, CDCl₃) δ1.54 (d, 3H), 2.92-3.10 (m, 2H), 3.59 (dd, 1H), 4.51 (t, 2H), 5.51 (s, 1H), 5.63 (s, 1H)

M-A3: ¹H NMR (300 MHz, CDCl₃) δ3.18 (m, 8H), 3.24 (s, 8H), 5.29 (s, 6), 5.29 (m, 2H), 8.05 (s, 4H)

M-A14: ¹H NMR (300 MHz, CDCl₃) δ1.65 (m, 4H), 2.20 (t, 4H), 3.02 (d, 8H), 3.21 (s, 8H), 5.01 (m, 2H), 5.22 (s, 4H)

### Making of Intraocular Lens

### Example 1

In a sample vessel having a volume of 30 ml were put 100 g of Exemplified Compound M-15 as cyclic allylsulfide monomers represented by the formula (I), 5 g of Exemplified Compound M-A14 and 0.4 g of azoisobutyronitrile (AIBN) as a polymerization initiator. These ingredients were fully mixed with stirring, and thereby a homogeneous solution of monomer mixture was prepared.

This monomer mixture solution was poured into a polypropylene mold for making of intraocular lenses. The mold was placed in a pressure furnace for polymerization purposes, the solution was heated up to 100° C. in an atmosphere of nitrogen under a 2.5 kgf/cm² (0.245 MPa) of pressure, and subjected to polymerization reaction for 2 hours. Thus, the solution was made into an optic member (diameter: 6 mm, thickness: 0.6 mm) of the same shape as the optic part 11 of the intraocular lens 10 shown in FIG. 1.

In addition, the monomer mixture solution was polymerized in a separate box container, and thereby made into a sheet-form sample piece (length: 15 mm, width: 15 mm, thickness: 0.6 mm).

Each of the thus obtained samples was immersed in 100 ml of methanol, whereby monomers remaining unpolymerized therein were eliminated, and then evaluations described below were performed on the resulting samples.

### Examples 2 to 12 and Comparative Examples 1 to 11

Optic members and sheet-form sample pieces were made in the same manners as in Example 1, except that the chemical species and usages of the raw material monomers and the polymerization initiator were changed to those shown in Table 1, respectively.

TABLE 1

| | | (b) Polyfunctional Second Monomer | | | |
|---|---|---|---|---|---|
| | | | Other Polyfunctional | | |
| | (a) First Monomer | Formula (AI) | Monomer | Another Monomer | Polymerization Initiator |
| Example 1 | M-15 (100 g) | M-A14 (5 g) | — | | AIBN (0.4 g) |
| Example 2 | M-16 (100 g) | — | EDMA (5 g) | | V-65 (0.2 g) |
| Example 3 | M-16 (100 g) | — | EDMA (5 g) | T-150 (5 g) | V-65 (0.2 g) |
| | | | | HMPO (0.03 g) | |
| Example 4 | M-24 (100 g) | — | EDMA (5 g) | — | V-65 (0.2 g) |
| Example 5 | M-16 (25 g) | — | EDMA (5 g) | PEA (50 g) | AIBN (0.4 g) |
| | M-24 (25 g) | | | | |
| Example 6 | M-24 (50 g) | — | GAMA (1 g) | HPPA (50 g) | V-65 (0.2 g) |
| Example 7 | M-16 (50 g) | — | EDMA (5 g) | — | V-65 (0.2 g) |
| | M-24 (50 g) | | | | |
| Example 8 | M-16 (90 g) | — | EDMA (15 g) | — | V-65 (0.2 g) |
| Example 9 | M-16 (100 g) | — | DEDMA (5 g) | — | V-601 (0.2 g) |
| Example 10 | M-16 (75 g) | M-A3 (25 g) | EDMA (5 g) | — | V-65 (0.2 g) |
| Example 11 | M-16 (75 g) | M-A3 (25 g) | EDMA (5 g) | T-150 (5 g) | V-601 (0.2 g) |
| | | | | HMPO (0.03 g) | |
| Example 12 | M-24 (75 g) | M-A3 (25 g) | EDMA (5 g) | — | AIBN (0.4 g) |
| Comparative | — | — | EDMA (5 g) | PEA (100 g) | AIBN (0.4 g) |
| Example 1 | | | | | |
| Comparative | — | — | GAMA (1 g) | HPPA (100 g) | V-65 (0.2 g) |
| Example 2 | | | | | |
| Comparative | — | — | GAMA (5 g) | HPPA (100 g) | V-65 (0.2 g) |
| Example 3 | | | | | |
| Comparative | — | — | GAMA (1 g) | HPPA (50 g), | V-65 (0.2 g) |
| Example 4 | | | | POEMA (50 g) | |
| Comparative | — | — | GAMA (5 g) | HPPA (50 g), | V-65 (0.2 g) |
| Example 5 | | | | POEMA (50 g) | |
| Comparative | — | — | GAMA (5 g) | HPPA (20 g), | V-65 (0.2 g) |
| Example 6 | | | | POEMA (40 g), | |
| | | | | BA (40 g) | |
| Comparative | M-15 (100 g) | — | — | — | AIBN (0.4 g) |
| Example 7 | | | | | |
| Comparative | M-16 (100 g) | — | — | — | V-65 (0.2 g) |
| Example 8 | | | | | |
| Comparative | M-24 (100 g) | — | — | — | V-65 (0.2 g) |
| Example 9 | | | | | |
| Comparative | M-16 (100 g) | — | — | T-150 (5 g) | V-65 (0.2 g) |
| Example 10 | | | | HMPO (0.03 g) | |
| Comparative | M-16 (50 g) | — | — | PEA (5 g) | V-65 (0.2 g) |
| Example 11 | M-24 (50 g) | | | | |

Compounds corresponding to the abbreviations in Table 1, respectively, are as follows.
EDMA: Ethylene glycol dimethacrylate (produced by Wako Pure Chemical Industries, Ltd)
DEDMA: Diethylene glycol dimethacrylate (produced by Wako Pure Chemical Industries, Ltd)
T-150: 2-(2′-Hydroxy-3′-tetrabutyl-5′-methylophenyl)-5-(2′-methacryloxymethyl)benzotriazole
HMPO: 4-(5-Hydroxy-3-methyl-1-phenyl-4-pyrazolylmethylene)-3-methacrylamino-1-phenyl-2-pyrazoline-5-one
GAMA: 2-Hydroxy-1-acryloxy-3-methacryloxypropane (synthetic compound)
PEA: 2-Phenylethyl acrylate (produced by Wako Pure Chemical Industries, Ltd)
HPPA: 2-Hydroxy-3-phenoxypropyl acrylate (produced by Wako Pure Chemical Industries, Ltd)
POEMA: 2-Phenoxyethyl methacrylate (produced by Sigma-Aldrich Corporation)
BA: Butyl acrylate (produced by Wako Pure Chemical Industries, Ltd)
AIBN: Azobisisobutyronitrile (produced by Wako Pure Chemical Industries, Ltd)
V-65: 2,2′-Azobis(2,4-dimethylvaleronitrile) (produced by Wako Pure Chemical Industries, Ltd)
V-601: Dimethyl 2,2′-Azobis(2-methylpropionate) (produced by Wako Pure Chemical Industries, Ltd)

Additionally, when each of the samples prepared in Examples 1 to 6 and 8 to 11 was subjected to 6-hour Soxhlet extraction using a good solvent, such as acetone or methyl ethyl ketone, and the extract thus obtained was examined for unreacted monomers by means of a gas chromatograph-mass spectrometer, the total amount of the unreacted monomers therein was found to be 50 ppm or below.

When the total amount of unreacted monomers in each of the samples obtained in Comparative Examples 1 to 11 was determined in the same way as mentioned above, it was found to be 50 ppm or below.

### <Evaluation>

Evaluations of the following categories were made on each of the samples made in Examples 1 to 6 and 8 toll and Comparative Examples 1 to 6.

### 1. Appearance

After each of the optic members made in the foregoing manners was immersed for 24 hours in water kept at 23° C., the lateral face thereof was exposed to light from a white lamp (LG-PS2, made by Olympus Corporation) and observed by the naked eye. By doing so, each optic member was checked for transparency and presence or absence of discoloration, and the appearance thereof was evaluated on the basis of the following criteria.

### Evaluation Criteria:

A: colorless and transparent

B: slightly clouded

C: clouded

### 2. Refractive Index

The refractive index of each of the optic members made in the foregoing manners at a wavelength of 546.1 nm (e-ray) was measured at 23° C. by means of a refractometer, DR-M2 made by ATAGO Co., Ltd.

### 3. Glistening

After each of the optic members made in the foregoing manners was immersed in 33° C. water for 24 hours and then immersed in 28° C. or 23° C. water, its appearance was observed under a stereoscopic microscope (U-PMTVC, made by Olympus Corporation) and evaluated on the basis of the following criteria.

### Evaluation Criteria:

A: During the water immersion, neither blistering nor clouding is developed even by the temperature change from 33° C. to 23° C. and excellent transparency is retained.

B: During the water immersion, slight blistering and clouding are perceived by the temperature change from 33° C. to 23° C., but by the temperature change 33° C. to 28° C. neither blistering nor clouding is perceived and excellent transparency is retained.

C: During the water immersion, serious blistering and clouding are perceived by the temperature change from 33° C. to 23° C. and even by the temperature change from 33° C. to 28° C. slight blistering and clouding are perceived.

D: During the water immersion, serious blistering and clouding are perceived even by the temperature change from 33° C. to 28° C.

E: Regardless of the temperature changes, blisters and white turbidity are present in a material from the beginning of water immersion, and the material is opaque.

TABLE 2

| | Appearance | Refractive Index | Glistening |
|---|---|---|---|
| Example 1 | A | 1.567 | B |
| Example 2 | A | 1.601 | A |
| Example 3 | A | 1.602 | A |
| Example 4 | A | 1.552 | A |
| Example 5 | A | 1.566 | B |
| Example 6 | A | 1.553 | B |
| Example 8 | A | 1.612 | A |
| Example 9 | A | 1.607 | A |
| Example 10 | A | 1.622 | A |
| Example 11 | A | 1.582 | A |
| Comparative | A | 1.559 | D |
| Example 1 | | | |
| Comparative | C | 1.554 | E |
| Example 2 | | | |
| Comparative | C | 1.559 | D |
| Example 3 | | | |
| Comparative | C | 1.562 | E |
| Example 4 | | | |
| Comparative | B | 1.568 | D |
| Example 5 | | | |
| Comparative | A | 1.533 | D |
| Example 6 | | | |

As can be seen from Table 2, the materials made in Examples 1 to 5 and 8 to 11 present no problem about their appearances during the water immersion and have physical properties suitable as intraocular lenses. In addition, their polymers have refractive indexes higher than 1.55 and are highly effective in inhibiting the occurrence of glistening. Although there is a previous finding that the glistening can be inhibited from occurring by addition of hydrophilic monomers, the monomers according to the invention are not such hydrophilic monomers, accordingly it has been shown that the materials according to the invention produced unexpected effects.

On the other hand, the materials made in Comparative Examples 1 to 6 have proved to be conspicuous for occurrence of glistening in particular.

By using curable resin compositions according to the invention, it is possible to make intraocular lens materials and intraocular lenses each having a high refractive index, excellent flexibility and transparency and ensuring reduction in occurrence of glistening. Therefore, each of the intraocular lenses according to the invention is especially suitable for use as a soft intraocular lens which can be folded and inserted into the eye through a small incision. Moreover, the curable resin compositions according to the invention allow sophisticated shape control because they cause a slight curing shrinkage in volume, and what's more they ensure speeding-up in curing of the resins at the time of lens making because their polymerization resists being inhibited by oxygen, and thereby the productivity can be increased.

The entire disclosure of each and every foreign patent application from which the benefit of foreign priority has been claimed in the present application is incorporated herein by reference, as if fully set forth.

What is claimed is:

1. A curable resin composition for intraocular lens, comprising:
(a) at least one first monomer selected from compounds represented by following formula (I);
(b) a polyfunctional second monomer; and
(c) a radical polymerization initiator: 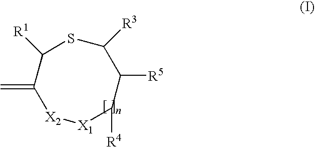
wherein R¹ represents a hydrogen atom or an alkyl group;
X₁ represents an oxygen atom or a sulfur atom;
when X₁ represents a sulfur atom, X₂ represents CR² where R² represents a hydrogen atom or an alkyl group;
when X₁ represents an oxygen atom, X₂ represents C═O;
R³ to R⁵ each independently represents a hydrogen atom, an alkyl group, an aryl group, a hetero ring group, an alkoxy group, an aryloxy group, an alkylthio group, an arylthio group, an alkoxycarbonyl group, an aryloxycarbonyl group, an acyloxy group, an arylcarbonyloxy group, an acylamino group, a sulfonylamino group, an amino group, an acyl group or a halogen atom; and
n represents 0 or 1.

2. The curable resin composition for intraocular lens according to claim 1,
wherein, in the formula (I),
X₁ represents a sulfur atom;
X₂ represents CR²; and
n represents 1.

3. The curable resin composition for intraocular lens according to claim 1,
wherein the polyfunctional second monomer (b) is at least one selected from the group consisting of compounds represented by following formula (AI), polyfunctional methacrylate monomers and polyfunctional acrylate monomers: 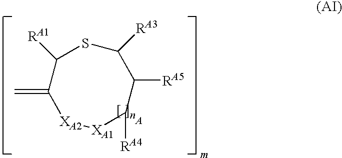
wherein R^{A1} represents a hydrogen atom or an alkyl group;
X_{A1} represents an oxygen atom or a sulfur atom;
when X_{A1} represents a sulfur atom, X_{A2} represents CR^{A2} where R^{A2} represents a hydrogen atom or an alkyl group;
when X_{A1} represents an oxygen atom, X_{A2} represents C═O;
R^{A3} to R^{A5} each independently represents a hydrogen atom, an alkyl group, an aryl group, a hetero ring group, an alkoxy group, an aryloxy group, an alkylthio group, an arylthio group, an alkoxycarbonyl group, an aryloxycarbonyl group, an acyloxy group, an arylcarbonyloxy group, an acylamino group, a sulfonylamino group, an amino group, an acyl group or a halogen atom;
n_{A} represents 0 or 1;
m represents 2 to 6; and
the formula (AI) represents a dimer to hexamer which bonds via any of R^{A1} to R^{A5}.

4. The curable resin composition for intraocular lens according to claim 1, which contains the polyfunctional second monomer (b) in an amount of from 0.05 to 40 mass % with respect to a total amount of monomers contained in the curable resin composition.

5. The curable resin composition for intraocular lens according to claim 1, further comprising:
a monomer having ultraviolet absorbing power in an amount of from 0.05 to 8 mass % with respect to a total amount of monomers contained in the curable resin composition.

6. The curable resin composition for intraocular lens according to claim 1, further comprising:
a monomer having yellow coloring power in an amount of 0.0001 to 0.5 mass % with respect to a total amount of monomers contained in the curable resin composition.

7. An intraocular lens material obtained by polymerization reaction of the curable resin composition for intraocular lens according to claim 1.

8. An intraocular lens, comprising:
an optic part; and
a supporting part that fixes the optic part to an appropriate place in the eye,
wherein the optic part is formed from the intraocular lens material according to claim 7.
